(19) United States
(12) Patent Application Publication
(10) Pub. No.: US 2010/0196445 A1
(43) Pub. Date: Aug. 5, 2010

(54) SYSTEMS AND METHODS FOR DELIVERY OF BIOLOGICALLY ACTIVE AGENTS

(76) Inventors: Nathaniel E. David, Mill Valley, CA (US); Tahir A. Mahmood, La Jolla, CA (US); Tobin Dickerson, San Diego, CA (US)

Correspondence Address: WILSON, SONSINI, GOODRICH & ROSATI, 650 PAGE MILL ROAD, PALO ALTO, CA, 94304-1050, US

(21) Appl. No.: 12/617,511

(22) Filed: Nov. 12, 2009

Related U.S. Application Data
(60) Provisional application No. 61/113,975, filed on Nov. 12, 2008.

(51) Int. Cl. A61K 9/70; A61K 38/16; A61K 38/46; A61K 38/45; A61K 38/47; A61K 38/48; A61K 39/395; A61P 43/00; A61M 5/00

(52) U.S. Cl. 424/443; 514/12; 424/94.65; 424/94.62; 424/94.5; 424/94.61; 424/94.64; 424/94.63; 424/134.1; 604/265

(57) ABSTRACT

The invention provides methods and devices for the delivery of therapeutic or biologically active agents to tissue, for example by facilitating the transport of said agents through the skin of a human or animal. Therapeutic agents include various botulinum neurotoxin (BoNT) and other biologically active agents, which can be delivered across the skin and into the dermis using multiple strategies including microneedle drug delivery, transport moieties, or penetration enhancers such as extracellular matrix-digestive enzymes.

### CROSS-REFERENCE

This application claims the benefit of U.S. Provisional Application No. 61/113,975, filed Nov. 12, 2008, which application is incorporated herein by reference.

### BACKGROUND OF THE INVENTION

Delivery of biologically active agents through tissue barriers such as skin remains a challenging problem. A common such therapeutic agent is botulinum neurotoxin (“BTX”), which is in wide use for a variety of indications and for aesthetic use. Botulinum neurotoxins can be injected intramuscularly, subcutaneously, or intradermally. Roughly a dozen painful injections may be required to treat facial wrinkles, and more than a hundred painful intra-dermal injections are required to treat excessive sweating of the underarms (axilla), palms, or feet. Effective strategies for delivery of botulinum neurotoxins into skin and muscle without the use of a hypodermic needle are therefore highly desirable. This invention provides methods and devices for transdermal delivery of biologically active agents, such as botulinum neurotoxins and other therapeutic agents.

### SUMMARY OF THE INVENTION

In one aspect, the invention provides a microneedle device comprising one or more microneedles that: (a) is coated with up to 10, 50, 100, 500, 1000, 2000, 4000, 10000, or 50000 units of a botulinum neurotoxin; (b) contains up to 10, 50, 100, 500, 1000, 2000, 4000, 10000, or 50000 units of a botulinum neurotoxin; or (c) is effective to deliver up to 10, 50, 100, 500, 1000, 2000, 4000, 10000, or 50000 units of a botulinum neurotoxin per injection, wherein the microneedle device (a) is coated with a formulation comprising a botulinum neurotoxin and an enzyme system which digests an extracellular matrix; (b) contains within the needle a formulation comprising an enzyme system that digests an extracellular matrix and a botulinum neurotoxin; or (c) is effective to deliver an enzyme that digests an extracellular matrix and a botulinum neurotoxin. In one embodiment of the microneedle device, the botulinum neurotoxin is associated with a targeting moiety, such as a targeting moiety comprises a transport moiety. In an embodiment of microneedle devices of the invention, the botulinum neurotoxin resides essentially only on the tip of the one or more microneedles. In another embodiment, the microneedle device is dissolvable.

The invention further provides a method for treating a skin region having wrinkles comprising: (a) applying a microneedle device to the skin region to perforate the skin region; (b) applying to the skin region a penetration enhancer; and (c) applying to the skin region a botulinum neurotoxin. In one embodiment, steps (b) and (c) occur simultaneously. In another embodiment, steps (a), (b) and (c) occur simultaneously. In still another embodiment, step (b) and/or (c) occurs using a topical patch. Alternatively, step (b) and/or (c) occurs using a subcutaneous injection, or step (b) and/or step (c) are performed using a microneedle device, or step (b) and/or (c) are performed using topical administration of a foam or gel. In yet another embodiment, step (a) occurs prior to step (b) and step (c). In some embodiments, step (a), (b) or (c) are repeated at least 2, 3, 4, 5, 6, 7, or 8 times. For instance, steps (a), (b) and (c) are repeated at least 2, 3, 4, 5, 6, 7, or 8 times. The repetition is, for example, per month or per year. Step (a), (b), or (c) may be applied for at least 20 seconds, 25 seconds, 30 seconds or 60 seconds, for example the administering step occurs for at least at least 20 seconds, 25 seconds, 30 seconds or 60 seconds. The microneedle device may also comprise dissolvable microneedles.

In an embodiment of the method of the invention, the penetration enhancer comprises an enzyme that degrades extracellular matrix. For example, the enzyme is papain, hyaluronidase, streptokinase, streptodornase, trypsin, chymotrypsin, alpha-chymotrypsin, alpha-amylase, deoxyribonuclease, collagenase, sutilain, or any mixture thereof. In some embodiments, the step of applying a penetration enhancer comprises applying papain, Azone™, and propylene glycol.

In another aspect, the invention provides a method for treating a skin region having wrinkles comprising administering to the skin region less than 100, 500, 1000, 2000, 4000 or 10000 units of a botulinum neurotoxin. The invention also provides a method for treating a patient suffering from hyperhidrosis by administering to a skin region of the patient less than 100, 500, 1000, 2000, 4000 or 10000 units of a botulinum neurotoxin. For example, the skin region is a forehead, a palm, glabella, or nasolabial fold, or alternatively is the palm or axilla. In one embodiment, the administering is performed by applying a microneedle device to the skin region. In another embodiment, the method further comprises administering to the skin region an enzyme that digests an extracellular matrix. The skin region may be perforated by applying to it a microneedle device. In some embodiments, the administering step occurs for at least 20, 25, 30 or 60 seconds.

In another aspect, the invention provides an injectable formulation consisting essentially of a botulinum neurotoxin and a penetration enhancer system. In one embodiment, the penetration enhancer is an enzyme that degrades extracellular matrix including but not limited to papain, hyaluronidase, streptokinase, streptodornase, trypsin, chymotrypsin, alpha-chymotrypsin, alpha-amylase, deoxyribonuclease, collagenase, sutilain, or any mixture thereof. The penetration enhancer system optionally comprises papain and optionally Azone™ and propylene glycol.

The botulinum neurotoxin for use in the methods, devices and formulations of the invention may be serotype A, B, C1, D, E, F, or G. In some embodiments, the botulinum neurotoxin is associated with a transport moiety, such as a targeting moiety which comprises a polyarginine. For example, the invention provides an injectable formulation comprising a botulinum neurotoxin associated with a targeting moiety, wherein the targeting moiety comprises a polyarginine.

The invention also provides a targeting moiety construct comprising: (a) a transport moiety for delivering a load across the skin barrier; and (b) a botulinum neurotoxin binding moiety coupled to said transport moiety. In some embodiments, the construct comprises a second transport moiety coupled to a different position of said botulinum neurotoxin binding moiety. Transport moieties include, but are not limited to, dendrimers and polyarginines. Botulinum neurotoxin binding moieties are, for example, sequences derived from SV2 (amino acids 518-580), Syt II (amino acids 40-60), and peptides or antibodies such as botulinum neurotoxin binding antibodies. The construct also includes, in some embodiments, a flexible linker, for example a Gly-Ser linker.

In still another aspect of the invention, a method is provided for making a microneedle device comprising: (a) providing a plurality of integrated microneedles; (b) coating at least one or more of the microneedles with a botulinum neurotoxin or an extracellular matrix degrading enzyme; and (c) optionally further contacting at least one or more of the microneedles with a penetration enhancer. In one embodiments, the microneedles are degradable. In another embodiment, the penetration enhancer is selected from the group consisting of: papain, hyaluronidase, streptokinase, streptodornase, trypsin, chymotrypsin, alpha-chymotrypsin, alpha-amylase, deoxyribonuclease, collagenase, sutilain, or any mixture thereof.

Kits are also provided comprising a microneedle device coated with a botulinum neurotoxin system and a penetration enhancer. Microneedle devices include, for example, one or more porous microneedles or one or more non-porous microneedles. A microneedle device may also comprise a reservoir attached to one or more microneedles. In some embodiments, dissolvable microneedles are used. Penetration enhancers include enzyme systems that digest extracellular matrix, for example a protease or a hyaluronidase. Proteases can be, for example, matrix metalloproteases, thiol proteases or serine proteases. Botulinum neurotoxins for use in the kit are, for example, serotype A or B. In some embodiments, the botulinum neurotoxin is BOTOX™. For example, the microneedle device is configured for delivering at least 4, 50, 100, 200, 500 or 1,000 Units of BOTOX™. In one embodiment, either the enzyme system or the botulinum neurotoxin system is coated on a subset of the microneedles. Alternatively, either the enzyme system or the botulinum neurotoxin system is within a subset of the microneedles, or the botulinum neurotoxin resides essentially only on the tip of the microneedle(s) in the microneedle device. In other embodiments, the botulinum neurotoxin system comprises a botulinum neurotoxin associated with a targeting moiety, such as a transport moiety. For example, the transport moiety is poly-arginine.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the lethality of botulinum neurotoxin administered via a microneedle device of the invention, in the presence or absence of hyaluronidase.

FIG. 2 describes the effect of administration of hyaluronidase on the speed of botulinum neurotoxin action when administered via a microneedle device.

FIG. 3 illustrates a general strategy for preparing targeting moieties according to the invention.

FIG. 4 shows additional possible configurations for targeting moieties of the invention.

FIGS. 5-7 describe several possible nucleotide and peptide sequences of targeting moieties of the invention.

### DETAILED DESCRIPTION OF THE INVENTION

The invention provides methods, devices and formulations for delivery of biologically active agents across tissue membranes such as skin. Biologically active agents include neurotoxins, such as, e.g., botulinum toxin.

In one aspect, the invention provides a microneedle device comprising one or more microneedles that is coated with, contains, or is effective to deliver a neurotoxin (e.g., botulinum neurotoxin), wherein the microneedle device: (a) is coated with a formulation comprising an enzyme system which digests an extracellular matrix; (b) contains within the needle a formulation comprising an enzyme that digests an extracellular matrix and a botulinum neurotoxin; or (c) is effective to deliver an enzyme that digests an extracellular matrix.

The invention contemplates the use of microneedle technology for delivery of the neurotoxin or neurotoxin formulation. Microneedle delivery technologies can facilitate drug delivery to various skin depths using arrays of short (e.g., <4 mm in length) needle(s). Such microneedles pierce the stratum corneum and underlying layers of the skin to present drug directly into the epidermis, dermis, or subcutaneous space. Due to the small size of microneedles, application is virtually pain-free, with minimal (if any) bleeding or application site reaction. Herein, the term “microneedle” refers to an elongated structure that is sufficiently long to penetrate through the stratum corneum skin layer and into the epidermal/dermal layer, but sufficiently short to not result in substantial pain due to activation of nerve endings.

Microneedles that facilitate transdermal delivery are described in Prausnitz, Advanced Drug Delivery Reviews 56 (2004) 581-587; Zahn et al., Biomed. Microdevices 6 (2004) 183-190; Shirkhandeh J. Materials Sci. 16 (2005) 37-45; Park, J. Controlled Release 104 (2005) 51-66; U.S. Pat. Nos. 3,964,482, 6,503,231, 6,745,211; 6,611,707; 6,334,856; and U.S. Published Patent Application Nos. 2005/0,209,565, 2004/0,106,904, 2004/0,186,419, and 2002/0,193,754. Suitable microneedles have been fabricated from many materials, including silicon, metals, and polymers. Davis et al. describe the mechanics of microneedle insertion into the skin (Davis et al., J. Biomech. 37 (2004) 1155-1163).

Solid or hollow microneedles can be used in the embodiments described herein. In one embodiment, the microneedles for use in the invention are solid. For example, channels can be made by penetrating the skin with a microneedle array, followed by removal of the needles and subsequent application of the drug (see, e.g., Martanto et al., Pharm. Res. 21 (2004) and McAllister et al., PNAS 100 (2003) 13755-13760). The formulation comprising therapeutic agent according to the invention may be applied to the microneedle-treated site as a gel, hydrogel, topical cream, salve, ointment, or other topical formulation; and/or by using delivery devices such as bandages, occlusive bodies, patches, and/or the like.

In another embodiment, solid (non-porous) microneedles are coated with a formulation according to the invention prior to application to the skin. The skin is then punctured using the microneedles, which are kept in contact with the skin surface for a sufficient period of time, allowing diffusion of the therapeutic agent into the surrounding tissue. (Prausnitz, Advanced Drug Delivery Reviews 56 (2004) 581-587). In another embodiment, hollow (porous) microneedles are used, which contain channels that allow storage of the formulations of the invention comprising therapeutic agent. Upon application to skin, the therapeutic agent diffuses into tissue by diffusion or pressure-driven flow. (Zahn et al., Biomed. Microdevices 6 (2004) 183-190).

Microneedles can be used alone or as arrays of more than one microneedle. Various sizes of arrays are suitable for use with the invention. In one embodiment, 1-10 microneedles are used. In other embodiments, a microneedle array comprising 10-25, 10-50, 25-50, 25-200, 25-100, or 50-100 needles is used. An array of microneedles can vary on the basis of several factors, including but not limited to, length, diameter, interneedle distance, sharpness, and the total number of microneedles used. In an exemplary embodiment, an array of microneedles comprises a 10×10 matrix. In another embodiment, an array of microneedles comprises a 20×20 matrix. In some embodiments, the distance between each microneedle in an array is from approximately 100 μm to approximately 400 μm. In an embodiment, the particular dimensions of the array can be chosen depending on the desired enhancement of skin permeability.

In some embodiments, a microneedle has a length from 20 μm to approximately 1000 μm, for example from approximately 50 μm to approximately 150 μm, or from approximately 150 μm to approximately 500 μm, or from 500 μm to approximately 1000 μm, or from 600 μm to approximately 800 μm. In other embodiment, a microneedle is approximately 50, 100, 250, 500, 600, 700, 800, 900 or 1000 μm in length. In still other embodiments, the microneedle is at least 50, 100, 250, 500, 600, 700, 800, 900 or 1000 μm in length. In other embodiments, the microneedle is less than 50, 100, 250, 500, 600, 700, 800, 900 or 1000 μm in length. In one embodiment, the microneedle is approximately 700 μm in length. In some embodiments, the microneedle penetrates skin at a depth of approximately 400 μm to approximately 700 μm. In an embodiment, the microneedle penetrates skin at a depth approximately corresponding to the bottom of the stratum corneum. In another embodiment, the microneedle penetrates skin to approximately the top of the dermal layer. In still other embodiments, the microneedle penetrates skin up to a depth approximately in between the bottom of the stratum corneum and the top of the dermal layer. The microneedle may be any of a variety of diameters as needed to maintain efficacy. In some embodiments, the outer diameter of a microneedle can be from approximately 20 μm to approximately 100 μm. In other embodiments, the outer diameter of a microneedle can be from approximately 10 μm to approximately 50 μm. The inner diameter of a hollow microneedle can be from approximately 5 μm to approximately 70 μm in some embodiments. In addition the outer or inner diameters of a microneedle can be up to 10, 20, 30, 40, 50, 60, 70, 80, 90, or 100 μm. Any combination of the above microneedle dimensions may be used as necessary with the systems and methods described herein.

A microneedle can be manufactured from a variety of materials, including but not limited to, silicon, a metal, a polymer, and glass. In some embodiments, silicon microneedles are used. Silicon microneedles, whether solid or hollow, can be etched from silicon wafers. For example, the location of each microneedle is marked and the surrounding silicon is etched away, resulting in an array of microneedles attached to a common base. In some embodiments, the thickness of a silicon wafer is between approximately 300-600 μm.

In other embodiments, microneedles are made of a metal, including but not limited to nickel, titanium, and alloys such as stainless steel. In some embodiments, metal microneedles are made from epoxy molds which are then electroplated with a chosen metal, while the epoxy mold is subsequently etched away. The resulting microneedles may either be reusable or disposable. Microneedles may also be obtained from commercial sources, including Zosano Pharma, Inc.

The therapeutic agents can be applied to microneedles using a variety of methods. In one embodiment, a solution comprising the therapeutic agent is prepared and the solution is deposited onto/within the microneedles, followed by drying of the solution. Alternatively, the microarray is dipped into a solution comprising the therapeutic agent, resulting in coating of the microneedles with the therapeutic agent. When additional proteins or components are to be coated onto or within the microneedles of the invention, additional coating steps may be performed. Alternatively, several or all components of the solution are mixed into one solution which is then deposited onto/within the microneedle. Dip coating, spray coating, or other techniques known in the art may be used, for example those described in PCT Pub. No. WO 2006/138719. Coatings may be solid or semi-solid. In some embodiments, a therapeutic agent formulation comprises a suspension of microparticles or nanoparticles for controlled release of the therapeutic agent.

Generally, the therapeutic agents of the invention for use in such coatings are used as pharmaceutical compositions comprising pharmaceutically acceptable carriers, diluents, and/or excipients. Such carriers, diluents and/or excipients may not intended to have biological activity themselves, and are selected so as not to affect the biological activity of the therapeutic agent and the penetration enhancer, and may include distilled water, saline, physiological phosphate-buffered saline, Ringer's solutions, dextrose solution, and/or Hank's solution. Additional formulations and dosage forms to be used in conjunction with microneedle administration are described further below.

The present invention contemplates the use of neurotoxins. Neurotoxins include botulinum neurotoxins. The term “botulinum neurotoxin” or botulinum toxin” is used interchangeable. A neurotoxin (e.g., botulinum neurotoxin) of the invention can be purified or recombinant. It can be isolated as pure toxin or in a complex of the toxin associated with non-toxin proteins. The invention also contemplates the use of active fragments of the toxin which provide the same effect of the full toxin.

In one example, the invention contemplates the use of botulinum neurotoxin. Botulinum neurotoxin is a potent polypeptide neurotoxin produced by the anaerobic, gram positive bacterium Clostridium botulinum, which causes a neuroparalytic illness in humans and animals known as botulism. Symptoms of botulinum neurotoxin intoxication can progress from difficulty walking, swallowing, and speaking to paralysis of the respiratory muscles and death. Botulinum neurotoxin type A is a lethal natural biological agent, and it is believed that about 50 picograms of commercially available botulinum neurotoxin type A (as a purified neurotoxin complex) has a LD50 in mice of 0.3 ng (i.e. 1 unit). One unit of BOTOX™, available from Allergan, Inc., of Irvine, Calif., contains about 50 picograms (about 56 attomoles) of botulinum neurotoxin type A complex. One unit (U) of botulinum neurotoxin is defined as the LD50 upon intraperitoneal injection into female Swiss Webster mice weighing 18 to 20 grams each.

Several distinct botulinum neurotoxins are known as botulinum neurotoxin serotypes A, B, C1, D, E, F and G. Each of these serotypes is distinguished by neutralization with type-specific antibodies, and they generally vary in the animal species that they affect and in the severity and duration of the paralysis they evoke. For example, it has been determined that botulinum neurotoxin type A is more potent, as measured by the rate of paralysis produced in the rat, than botulinum neurotoxin type B. (Moyer E et al., “Botulinum neurotoxin Type B: Experimental and Clinical Experience”, Ch. 6, p. 71-85 of “Therapy With Botulinum neurotoxin”, eds Jankovic, J. et al. (1994)). Botulinum neurotoxin is known to bind with high affinity to cholinergic motor neurons, translocate into the neuron and block the release of acetylcholine. Additional uptake can take place through low affinity receptors, as well as by phagocytosis and pinocytosis.

For all toxin serotypes, the molecular mechanism of action appears to be similar and to involve at least three steps or stages. First, the toxin binds to the presynaptic membrane of the target neuron through a specific interaction between the heavy chain (H chain) and a cell surface receptor. The carboxyl end segment of the H chain appears to be important for targeting of the toxin to the cell surface. In the second step, the toxin crosses the plasma membrane of the affected cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, FIN, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm. Finally, the disulfide bond joining the heavy chain (H chain) and the light chain (L chain) is reduced. The toxic activity of botulinum neurotoxin is believed to be due to the L chain of the holotoxin, which is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Botulinum neurotoxin serotype A and E cleave the 25 kiloDalton (kD) synaptosomal associated protein SNAP-25. Botulinum neurotoxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum neurotoxin serotype C1 was originally thought to cleave syntaxin, but was found to cleave syntaxin and SNAP-25. Each of the botulinum neurotoxins specifically cleaves a different bond, except botulinum neurotoxin type B (and tetanus toxin) which cleave the same bond. Each of these cleavages block the process of vesicle-membrane docking, thereby preventing exocytosis of vesicle content. These differences in mechanism of action may affect the relative potency and/or duration of action of the various botulinum neurotoxin serotypes.

Botulinum neurotoxins have been used in clinical settings for the treatment of neuromuscular disorders characterized by hyperactive skeletal muscles (i.e. motor disorders). In 1989 a botulinum neurotoxin type A complex has been approved by the U.S. Food and Drug Administration for the treatment of blepharospasm, strabismus and hemifacial spasm. Subsequently, a botulinum neurotoxin type A was also approved by the FDA for the treatment of cervical dystonia and for the treatment of glabellar lines, and a botulinum neurotoxin type B was approved for the treatment of cervical dystonia. Clinical effects of peripheral intramuscular botulinum neurotoxin type A are usually seen within one week of injection. The typical duration of symptomatic relief from a single intramuscular injection of botulinum neurotoxin type A averages about three months, although significantly longer periods of therapeutic activity have been reported.

The molecular weight of the botulinum neurotoxin protein molecule, for all seven of the known botulinum neurotoxin serotypes, is about 150 kD. The botulinum neurotoxins are released by Clostridial bacterium as complexes comprising the 150 kD botulinum neurotoxin protein molecule along with associated non-toxin proteins. Thus, the botulinum neurotoxin type A complex can be produced by Clostridial bacterium as 900 kD, 500 kD and 300 kD forms. Botulinum neurotoxin types B and C1 is apparently produced as only a 700 kD or 500 kD complex. Botulinum neurotoxin type D is produced as both 300 kD and 500 kD complexes. Finally, botulinum neurotoxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum neurotoxin molecule comprise the relevant neurotoxin complex) may act to provide stability against denaturation to the botulinum neurotoxin molecule and protection against digestive acids when toxin is ingested. Additionally, the larger (greater than about 150 kD molecular weight) botulinum neurotoxin complexes may result in a slower rate of diffusion of the botulinum neurotoxin away from a site of intramuscular injection of a botulinum neurotoxin complex.

Botulinum neurotoxin type A can be obtained by establishing and growing cultures of Clostridium botulinum in a fermenter and then harvesting and purifying the fermented mixture in accordance with known procedures. All the botulinum neurotoxin serotypes are initially synthesized as inactive single chain proteins which must be cleaved or nicked by proteases to become neuroactive. The bacterial strains that make botulinum neurotoxin serotypes A and G possess endogenous proteases and serotypes A and G can therefore be recovered from bacterial cultures in predominantly their active form. In contrast, botulinum neurotoxin serotypes C1, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum neurotoxin type B serotype only cleave a portion of the toxin produced. The exact proportion of nicked to unnicked molecules depends on the length of incubation and the temperature of the culture. Therefore, a certain percentage of any preparation of, for example, the botulinum neurotoxin type B toxin is likely to be inactive. The presence of inactive botulinum neurotoxin molecules in a clinical preparation will contribute to the overall protein load of the preparation, which has been linked to increased antigenicity, without contributing to its clinical efficacy. Additionally, it is known that botulinum neurotoxin type B has, upon intramuscular injection, a shorter duration of activity and is also less potent than botulinum neurotoxin type A at the same dose level.

The Schantz process can be used to obtain crystalline botulinum neurotoxin type A, as set forth in Schantz, E. J., et al, Properties and use of Botulinum neurotoxin and Other Microbial Neurotoxins in Medicine, Microbiol Rev. 56; 80-99:1992. Generally, the botulinum neurotoxin type A complex can be isolated and purified from an anaerobic fermentation by cultivating Clostridium botulinum type A in a suitable medium. The known process can also be used, upon separation out of the non-toxin proteins, to obtain pure botulinum neurotoxins. Additionally, botulinum neurotoxins and/or botulinum neurotoxin complexes can be obtained from List Biological Laboratories, Inc., Campbell, Calif.; the Centre for Applied Microbiology and Research, Porton Down, U.K.; Wako (Osaka, Japan); Reloxin (Medicis); Metabiologics (Madison, Wis.); Sigma Chemicals of St. Louis, Mo.; and Simply Aesthetic (Singapore). Pure botulinum neurotoxin can also be used to prepare a pharmaceutical composition.

The biological activities of the botulinum neurotoxins (which are intracellular peptidases) are highly sensitive to heat, various chemicals surface stretching and surface drying. Dilution of the toxin complex obtained by the known culturing, fermentation and purification to the lower toxin concentrations used for pharmaceutical composition formulation results in rapid detoxification of the toxin unless a suitable stabilizing agent is present. Since the toxin may need to be used months or years after formulation, the toxin can stabilized with a stabilizing agent such as albumin and gelatin. Such formulations can be in solid form (freeze-dried or lyophilized) or as stable liquid formulations (e.g. Myobloc™, available from Solstice Neuroscience Inc.)

A commercially available botulinum neurotoxin containing pharmaceutical composition is sold under the trademark BOTOX™ and is available from Allergan, Inc., of Irvine, Calif.). BOTOX™ consists of a purified botulinum neurotoxin type A complex, albumin and sodium chloride packaged in sterile, vacuum-dried form. The vacuum-dried product is stored in a freezer at or below −5 C. BOTOX™ can be reconstituted with sterile, non-preserved saline prior to intramuscular injection. Each vial of BOTOX™ contains about 100 units (U) of Clostridium botulinum neurotoxin type A purified neurotoxin complex, 0.5 milligrams of human serum albumin and 0.9 milligrams of sodium chloride in a sterile, vacuum-dried form without a preservative. To reconstitute vacuum-dried BOTOX™, sterile normal saline without a preservative (0.9% Sodium Chloride Injection) is used by mixing with the proper amount of diluent in an appropriate size container. Since BOTOX™ may be denatured by bubbling or similar violent agitation, the diluent is gently mixed with the toxin. BOTOX™ is preferably administered within four hours after the vial is removed from the freezer and reconstituted. During these four hours, reconstituted BOTOX™ can be stored in a refrigerator at about 2 C to about 8 C. Reconstituted, refrigerated BOTOX™ has been reported to retain its potency for at least about two weeks. Neurology, 48:249-53:1997.

Another commercially available botulinum neurotoxin source is Dysport™, available from Beaufour Ipsen, Porton Down, England. A botulinum neurotoxin type B preparation (MyoBloc™) is available from Solstice Neurosciences, Inc.

In some embodiments of the devices and methods of the invention, the botulinum neurotoxin is associated with a targeting moiety. A targeting moietycan be any compound or peptide that enhances the transport of the neurotoxin through a biological membrane. In one embodiment, a targeting moiety possesses a binding component which is capable of binding the therapeutic agent with sufficiently high affinity, and also possesses a membrane transduction component which facilitates the transport of the construct through the membrane. In another embodiment, the targeting moiety is a peptide sequence comprising a binding site for the therapeutic agent additionally linked to a transporter sequence which interacts with a cell membrane. For example, the binding site and the transporter sequence may be part of the same peptide sequence. In another embodiment, the therapeutic agent binding site is linked to the one or more transport moiety via a linker. Such a linker may comprise, for example, between 1 and 5, 10, 15 or 20 amino acids. In one embodiment, the linker is a flexible linker comprising one or more Glycine and/or Serine residues.

In one embodiment, the targeting moiety comprises a neurotoxin binding site. Any protein known to possess affinity to a neurotoxin or to botulinum neurotoxin binding can be used to generate such a binding site. In one embodiment, the botulinum neurotoxin is botulinum neurotoxin type A and the binding site is a peptide sequence derived from synaptic vesicle glycoprotein 2 (“SV2”). SV2 is a protein receptor which binds botulinum neurotoxin with high affinity and serves as a BoNT/A receptor, see, e.g., Min Dong et al., SV2 Is the Protein Receptor for Botulinum Neurotoxin A, Science (2006); S. Mahrhold et al, The Synaptic Vesicle Protein 2C Mediates the Uptake of Botulinum Neurotoxin A into Phrenic Nerves, 580(8) FEBS Lett. 2011-2014 (2006).

In another embodiment the targeting moiety comprises a binding site to botulinum neurotoxin type B and the binding site is a peptide sequence derived from synaptotagmin I or II (“Syt I” or “Syt II”, respectively). For example, the binding site is a peptide sequence derived from Syt II. Syt II serves as a BoNT/B receptor and as a BoNT/G receptor, see, e.g., Min Dong et al., supra, (2003); and Andreas Rummel et al., supra, (2004). Syt II of rat or human origin may be used. The mouse Syt II peptide sequence is provided as GenBank Accession No. NP_{—}033333.2, and the human Syt II peptide sequence is publicly available under GenBank Accession No. NP_{—}001129976.1. Similarly, the mouse Syt I peptide sequence is provided as GenBank Accession No NP_{—}033332.1, and the human Syt I peptide sequence is publicly available under GenBank Accession No. NP_{—}001129278.1. For murine/rat sequences, the botulinum neurotoxin B binding domain of Syt II is located between amino acids 40 and 60, and 37-57 for the corresponding human sequence. The binding domain of Syt I is between amino acids 32-52 for the mouse/rat sequence, and 33-53 for the human sequence.

In another embodiment, the therapeutic agent binding site is an antibody. For example, therapeutic agent binding site is an antibody capable of binding botulinum neurotoxin type A or B. Such antibodies are described, for example, in U.S. Pat. No. 5,932,449.

In some embodiments, the targeting moiety comprises a transport moiety which facilitates transfer of the targeting moiety through a cell membrane. The plasma membrane of cells consists of a lipid bilayer that presents a barrier to entry of macromolecules into the cell. In some embodiments, the membrane transduction component is a “protein transduction domain” or “PTD”, which refers to a domain (for example, a peptide or other polymer) that is able to confer membrane translocation activity a moiety to which it is linked. PTDs may be used for intracellular delivery of proteins, nucleic acids, small molecules (e.g., low molecular weight pharmaceutical agents), and particles (see, e.g., Zhao & Weissleder (2003) Med Res. Rev. 24(1):1-12). For example, the HIV-1 TAT peptide has been used extensively for directing the intracellular delivery of an assortment of cargo, including DNA, liposomes and macromolecules. The efficiency of translocation of PTDs is generally very high, leading to uptake of the PTD and its cargo in cells of all cell types (Joliot & Prochiantz (2004) Nat. Cell Biol. 6(3):189-96). PTDs are sometimes referred to as cell penetrating peptides (CPPs). A large variety of PTDs have been characterized, including, but not limited to, naturally occurring PTDs, artificial PTDs, and PTDs selected from random libraries (see, e.g., Joliot & Prochiantz (2004) Nat. Cell Biol. 6(3):189-96; Zhao & Weissleder (2003) Med Res. Rev. 24(1):1-12; Saalik et al. (2004) Bioconj. Chem. 15:1246-1253; each of which is herein incorporated by reference in its entirety). Other examples of PTDs that may be used in the compositions of the invention include endosomal releasing polymers, such as GALA, as described further below.

In some embodiments, the PTD used in the invention is a PTD of the human immunodeficiency virus (HIV-1) TAT protein. One of the most well-studied PTDs is the highly cationic 11 amino acid residue PTD (YGRKKRRQRRR, SEQ ID NO:1) from the HIV-1 TAT protein (Frankel & Pabo (1988) Cell 55:1189-93; Green & Loewenstein (1988) Cell 55:1179-88). In-frame fusion proteins containing the TAT sequence were shown to direct cellular uptake of proteins that retained their activity intracellularly (Nagahara et al. (1998) Nat. Med. 4:1449-52; Kwon et al. (2000) FEBS Lett. 485:163-7; Becker-Hapak et al. (2001) Methods 24:247-256; Jo et al. (2001) Nat. Biotechnol. 19:929-33; Xia et al. (2001) Nat. Biotechnol. 19:640-4; Cao et al. (2002) J. Neurosci. 22:5423-31; Joshi et al. (2002) Genesis 33:48-54; Kabouridis et al. (2002) J. Immunol. 169:2587-93; Peitz et al. (2002) Proc. Natl. Acad. Sci. U.S.A. 99:4489-94). Various studies employing TAT-fusion methodologies have demonstrated transduction in a variety of both primary and transformed mammalian and human cell types, including peripheral blood lymphocytes, diploid fibroblasts, keratinocytes, bone marrow stem cells, osteoclasts, HeLa cells, and Jurkat T-cells (Fawell et al. (1994) Proc. Natl. Acad Sci. U.S.A. 91:664-68; Nagahara et al. (1998) Nat. Med. 4:1449-52; Gius et al. (1999) Cancer Res. 59:2577-80; Vocero-Akbani et al. (1999) Nat. Med. 5:29-33; Vocero-Akbani et al. (2000) Methods Enzymol. 322:508-21; Vocero-Akbani et al. (2001) Methods Enzymol. 332:36-49; Becker-Hapak et al. (2001) Methods 24:247-256). Furthermore, in vivo intracellular delivery by injection of a TAT-b-gal fusion has been demonstrated (Schwarze et al. (1999) Science 285:1569-72; Barka et al. (2000) J. Histochem. Cytochem. 48:1453-60). Naturally occurring PTDs include the homeodomain of the Drosophila melanogaster protein Antennapedia (Lindsay (2002) Curr. Op. Pharmacol. 2:587-94; Derossi et al. (1994) J. Biol. Chem. 269:10444-50), HSV-1 VP22 (Bennett et al. (2002) Nat. Biotechnol. 20:20), and Buforin II (Park et al. (2000) Proc. Natl. Acad. Sci. U.S.A. 97:8245-50).

Synthetic PTDs are also known, such as transportan (Pooga et al. (1998) FASEB J. 12:67-77; Soomets et al. (2000) Biochim. Biophys. Acta 1467:165-76), polylysine, polyarginine, and polyhistidine (which can be positively charged based on the pH of the formulation). Arginine/guanine rich PTD sequences include KRRQRRR, RKKRRQR, RKKRRQRR from HIV-TAT, Arg4 (RRRR), Arg5 (RRRRR), d-Arg5 (RRRRR), Arg16 (RRRRRRRRRRRRRRRR), Yeast PRP (TRRNKRNRIQEQLNRK), phi21 (TAKTRYKAEEAELIAERR), lambdaN (MDAQTRRRERRAEKQAQWKAAN), FHV coat (RRRRNRTRRNRRRVR), Arg6 (RRRRRR), d-Arg6 (RRRRRR), BMV Gag (KMTRAQRRAAARRNRWTAR), HTLV-II Rex (TRRQRTRRARRNR), HIV-1 Rev (TRQARRNRRRRWRERQR) HIV-1 TAT(GRKKRRQRRRPPQ, RRRQRRKKR), d-HIV-1 TAT (RKKRRQRRR), Arg7 (RRRRRRR), d-Arg (RRRRRRR), N-Arg7 (RRRRRRR), Arg8 (RRRRRRRR), d-Arg8 (RRRRRRRR), Arg9 (RRRRRRRRR), and d-Arg9 (RRRRRRRRR). These and other sequences are described in Wender et al. (2000) Proc. Natl Acad. Sci. U.S.A. 97:13003-8; Futaki et al. (2001) J. Biol. Chem. 276:5836-40; Suzuki et al. (2002) J. Biol. Chem. 277:2437-43; and Wender et al. (2002) J. Am. Chem. Soc. 124:13382-3.

In one embodiment of the invention, the membrane transduction component of the targeting moiety is a synthetic PTD. For example, the component is polyarginine with a length of 6-15 amino acids.

The targeting moieties of the present invention are made via standard recombinant techniques in molecular biology, including chemical conjugation and modification methods. In some embodiments, a gene or polynucleotide encoding the biologically active protein is first cloned into a construct, e.g., a plasmid or other vector. Then, the oligonucleotides that encode the repeating units of the polypeptide portion are cloned into the construct through a ligation or multimerization scheme, in which the oligonucleotides are ligated together to form a polynucleotide that encodes the polypeptide portion. In this manner, the oligonucleotides are added to the gene or polynucleotide that encodes the protein portion, thereby producing the chimeric DNA molecule within the construct. As an option, the chimeric DNA molecule may be transferred or cloned into another construct that is a more appropriate expression vector. At this point, a host cell capable of expressing the chimeric DNA molecule is transformed with the chimeric DNA molecule. The transformation may occur with or without the utilization of a carrier, such as an expression vector. Then, the transformed host cell is cultured under conditions suitable for expression of the chimeric DNA molecule, resulting in the encoding of the protein conjugate. Methods for performing these and other steps useful in the present invention are well known. See, Joseph Sambrook, et al., Molecular Cloning: A Laboratory Manual, 2nd ed., 1.53 (Cold Spring Harbor Laboratory Press 1989).

The obtained targeting moieties may be purified via one or more techniques. Typically, purification entails combinations of individual procedures such as gel filtration, affinity purification, salt fractionation, ion exchange chromatography, size exclusion chromatography, hydroxyapatite adsorption chromatography, hydrophobic interaction chromatography and gel electrophoresis. Protein refolding steps can be used, as necessary, in completing configuration of the protein conjugate. High performance liquid chromatography (HPLC) is often useful for final purification steps. See, in general, Robert K. Scopes, Protein Purification: Principles and Practice (Charles R. Castor, ed., Springer-Verlag 1994) and Joseph Sambrook, Molecular Cloning: A Laboratory Manual, 2nd edition (Cold Spring Harbor Laboratory Press 1989). Examples of multi-step purification separations are also described in Baron, et al., Crit. Rev. Biotechnol. 10:179-90 (1990) and Below, et al., J. Chromatogr. A. 679:67-83 (1994). In one embodiment, the recombinant targeting moiety is expressed in-frame with an affinity tag such as a glutathione-S transferase or a poly-His tag. Such tags can be cleaved following purification if desired or the protein may be used as is.

In one embodiment, the targeting moiety is a polypeptide comprising one therapeutic agent binding site and one transduction domain. In another embodiment, the targeting moiety is a polypeptide comprising one therapeutic agent binding site and more than one transduction domain, for example two transduction domains. In another example, two transduction domains flank the therapeutic agent binding site. In a specific embodiment, a targeting moiety is a peptide comprising a botulinum neurotoxin binding site, such as a binding site for botulinum neurotoxin type B, in addition to one or more Syt II binding peptides expressed in-frame.

In some embodiments, the present invention contemplates the use of penetration enhancers in addition to the neurotoxin. For example, a neurotoxin can be co-formulated with a penetration enhancer to create a first formulation. The first formulation can then be applied onto or into a microneedle and delivered dermal to a region of interest. Thus, the invention provides microneedle devices coated with, comprising, or effective to deliver a penetration enhancer in addition to a therapeutic agent (e.g., neurotoxin). In other embodiments, the invention also provides an injectable formulation comprising a neurotoxin (e.g., botulinum neurotoxin) and a penetration enhancer. The term “penetration enhancer” includes any compound or protein which increases the permeability of skin and facilitates delivery of the therapeutic agent. Penetration enhancers include, for example, enzyme systems that break down extracellular matrix components. The term “enzyme system” refers to any enzyme whether independent or part of a complex with other compounds or proteins. In one embodiment, the penetration enhancer comprises an enzyme system which includes an enzyme that can break down extracellular matrix, resulting in improved tissue permeability.

Suitable penetration enhancers of the invention include proteases such as serine proteases, thiol proteases, or matrix metalloproteases. In one embodiment, the protease is papain, hyaluronidase, streptokinase, streptodornase, trypsin, chymotrypsin, alpha-chymotrypsin, alpha-amylase, deoxyribonuclease, collagenase, or sutilain. For example, the protease is hyaluronidase.

Hyaluronidases are a family of enzymes that degrade hyaluronic acid, which is a component of the interstitial barrier in tissues. By breaking down hyaluronic acid and lowering its viscosity, hyaluronidases increase the permeability of tissue, allowing faster and/or more effective delivery of, for example, therapeutic agents. Hyaluronidases available commercially include Hydase™ (supplied by PrimaPharm Inc.), which is approved by the FDA for use in humans. Other brand names of hyaluronidases include Vitrase (ISTA Pharmaceuticals) are Amphadase (Amphastar Pharmaceuticals). Hyaluronidases suitable for use in the invention include naturally isolated enzymes or recombinant sources such as Hylenex (sold by Halozyme Therapeutics).

The amount of penetration enhancer used may vary with the type of application. In some embodiments, the penetration enhancer is a hyaluronidase, and is administered in a dosage range from 0.001 units to 100 USP units (approximately equal to the amount of enzyme that will cause a change in A600 nm absorbance of 0.330 per minute at pH 5.7 at 37° C. (45 minute assay)). In other embodiments, approximately between 0.001 and 0.1 units are administered, or approximately between 0.1 and 1, or approximately between 1 and 5, or approximately between 5 and 10, or approximately between 10 and 20, or approximately between 20 and 50, or approximately between 50 and 100 units of a penetration enhancer (e.g., hyaluronidase or papin) is administered per application. For example, in some cases at least 0.001, 0.01, 0.1, 1, 2, 5, 10, 20, 50, 100, or 150 units of a penetration enhancer (e.g., hyaluronidase) are administered per application (e.g., using a microneedle device, patch, subqutaneous injections, etc.). In other embodiments, up to approximately 0.001, 0.01, 0.1, 1, 2, 5, 10, 20, 50, 100, or 150 units of a penetration enhancer (e.g., hyaluronidase) are administered per application.

In another aspect, the invention provides a method for treating a patient comprising (a) applying a microneedle device comprising one or more microneedles to a skin region of said to perforate the skin region; (b) applying to the skin region a penetration enhancer; and (c) applying to the skin region a therapeutic agent.

In one embodiment, the therapeutic agent is a neurotoxin such as e.g., botulinum neurotoxin. A variety of indications can be treated using the devices and compositions herein including but not limited to hyperhydrosis; muscular spasticity and contractures, which include palsy (e.g. cerebral palsy), facial contractures, dystonia, hemifacial spasms, tremors, spasticity (including spasticity resulting from multiple sclerosis), and opthalmologic conditions. In other embodiments, the invention is used for the treatment of wrinkles such as glabellar wrinkles, facial lines such as hyperkinetic facial lines, forehead frown lines, midfacial wrinkles, mouth wrinkles, neck lines and banding, chin creases; facial diskinesis (Meige syndrome, hemifacial spasm, aberrant regeneration of facial nerves, apraxia of eyelid opening); jaw dystonia, occupational dystonia, corneal ulceration; spasmodic dystonia; and headache.

The dosage used for each indication is controlled to result in the proper dose of neurotoxin being delivered. Insufficient dosage results in incomplete paralysis, while excessive dosage may result in complete paralysis of the affected muscle groups. The specific dosages used may vary depending on the condition being treated and the therapeutic regime. For example, treatment of neurogenic inflammation or hyperactive sweat glands may require less neurotoxin than conditions in which it is desirable to paralyze subdermal, hyperactive muscle. In some embodiments, the dosage administered to skin region of interest is between 1 and 50,000 units. In other embodiments, the dosage administered to the skin region of interest is between 1 and 25,000 units are used, or between 1 and 15,000, between 1 and 10,000, between 1 and 5,000, between 1 and 2,500, or between 1 and 1,000 units per administration. In other embodiments, the dosage used is between 1 and 750 units, or between 1 and 500 units, or between 1 and 250 units, or between 1 and 100 units. In still other embodiments, the dosage used is up to 20,000 units, less than 15,000 units, less than 10,000 units, less than 5,000 units, less than 2,500 units, less than 1,000 units, less than 750 units, less than 500 units, less than 250 units, less than 100 units, or less than 50 units per administration. In yet other embodiments, the dosage used is more than 20,000 units, more than 15,000 units, more than 10,000 units, more than 5,000 units, more than 2,500 units, more than 1,000 units, more than 750 units, more than 500 units, more than 250 units, more than 100 units, or more than 50 units per administration. In some embodiments, the dosage used is approximately 0.1, 0.5, 1, 5, 10, 25, 50, 100, 150, 250, 500, 750, 1,000, 2,500, 5,000, 10,000, 25,000, or 50,000 units. In some embodiments, the invention provides a method for treating a skin region having wrinkles comprising administering to the skin region less than 2,000 units of a botulinum neurotoxin, wherein the administering is performed by applying a microneedle device to the skin region.

Dosage forms of botulinum neurotoxins for administration to human patients include ointments, pastes, creams, lotions, gels, powders, solutions, and patches. First, the toxin is mixed under sterile conditions with pharmaceutically acceptable carriers, stabilizers and/or buffers as necessary. General considerations useful in formulating therapeutic agents such as botulinum neurotoxins are described in the art, including, for example, by Remington: The Science and Practice of Pharmacy 21^{st} ed., Lippincott Williams & Wilkins, 2005.

In some embodiments of the invention, transdermal formulations of toxin are used in the form of patches. Adhesive patches, are described, for example, in U.S. Pat. Nos. 6,010,715; 5,591,767; 5,008,110; 5,683,712; 5,948,433 and 5,965,154. Transdermal formulations can employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of therapeutic agents of the invention is accomplished by means of iontophoretic patches and the like. In certain embodiments, transdermal patches provide controlled delivery of the therapeutic agent. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

The methods and devices disclosed herein may also be used for administration of therapeutic agents other than botulinum neurotoxins resulting in reduced immunogenicity. Human skin is an immunogenic organ populated by a large number of immune surveillance cells, including macrophages, dendritic cell (DC) subsets, including dermal DCs and plasmacytoid DCs (pDCs), and T cell subsets, including CD4+T helper 1 (TH1), TH2 and TH17 cells, γδ T cells and natural killer T (NKT). Co-administration of a penetration enhancer such as an enzyme system capable of degrading extracellular matrix, for example hyaluronidase, via microneedle devices allows significant modulation of the amount of therapeutic agent proteins required to achieve a desired biological effect as compared to microneedles treated with therapeutic agent proteins alone, and therefore allows for antigenicity to be significantly reduced. In another embodiment, antigenicity is reduced because the immune cells will be exposed to the therapeutic protein for a shorter amount of time if the protein is co-administered with hyaluronidase.

### Kits/Articles of Manufacture

In another aspect, the invention further provides kits comprising a microneedle device coated with a botulinum neurotoxin system and a penetration enhancer. In some embodiments, such kits and articles of manufacture comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products Include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprising a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions is presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, devices or formulations provided herein are prepared, placed in an appropriate container, and labeled for treatment of an indicated condition.

### EXAMPLES

### Example 1

### Administration of BoNT/A Using Microneedles and Hyaluronidase

Microneedle arrays (3M Drug Delivery Systems) with an array configuration of 10×10 needles and a needle height of 700 microns were coated with three different protein combination solutions: BoNT/A (Metabiologics) (botulinum neurotoxin type A), BoNT/A in combination with bovine serum albumin (BSA), and BoNT/A in combination with hyaluronidase (HAse). The BSA and HAse concentrations were held constant (14 mg/ml for BSA and 30 mg/ml for HAse, both proteins were diluted in phosphate buffered saline), while the amount of BoNT/A varied between 400 LD50 units and 50,000 LD50 units.

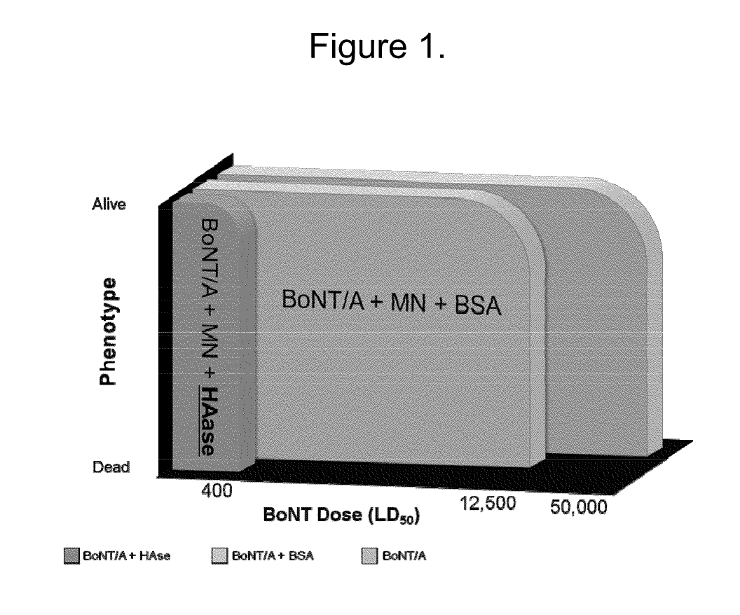

Using a pipettor, 30 μl of a protein solution was applied to an upside down array and the solution was allowed to air dry prior to use. The coated microneedle arrays were applied to the shaved hind legs of mice by applying pressure on the array such that the needles broke the epidermis and penetrated the dermal compartment of the skin. The microarrays were maintained on the hind legs for 10 seconds before removal. Once treated, the mice were returned to their cages with a distinctive marking on their tails to allow for identification. Mice were observed hourly for the first few hours and then twice daily, in the mornings and evenings. Treatment related deaths were recorded and the results of the BoNT/A dose-responses are shown in FIG. 1.

The results of the dose response showed that the inclusion of HAse on microneedle arrays alongside BoNT/A dramatically potentiated the biological effect of BoNT/A. About 120-fold less BoNT/A is required to cause death in mice after 24 hours when compared to BoNT/A alone. This result is not due to a protein effect as microneedle arrays coated with BoNT/A plus BSA only marginally improved the BoNT/A efficacy.

### Example 2

### Time-course of BoNT/A Activity Using a Microneedle Device of the Invention

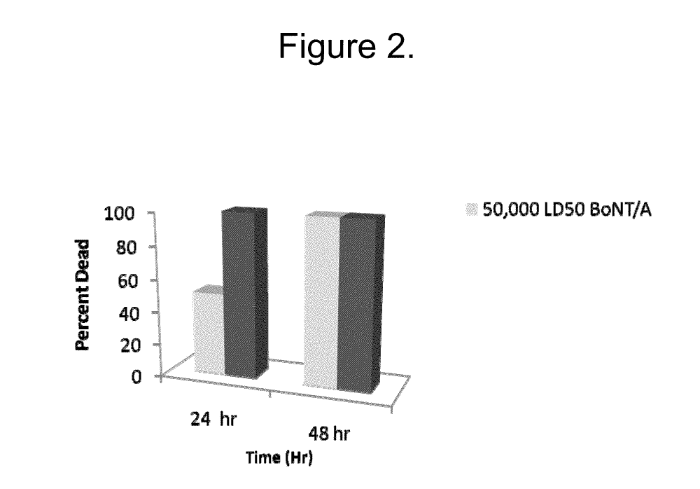

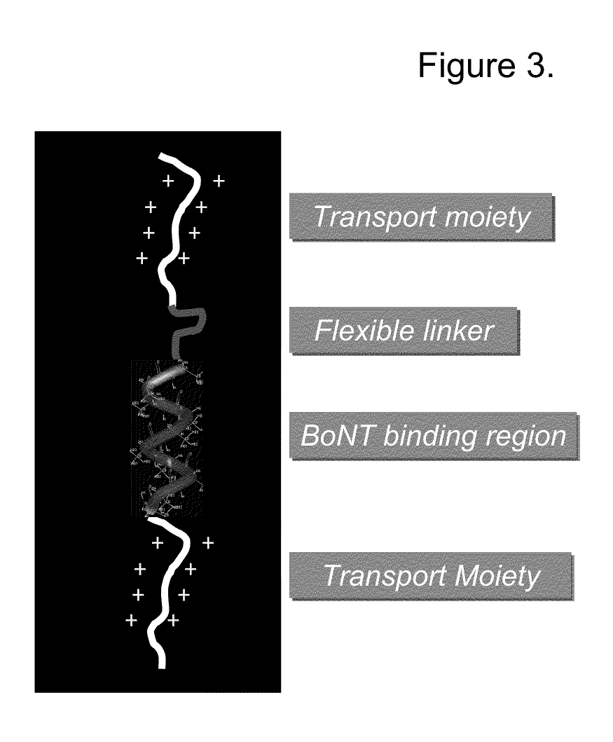

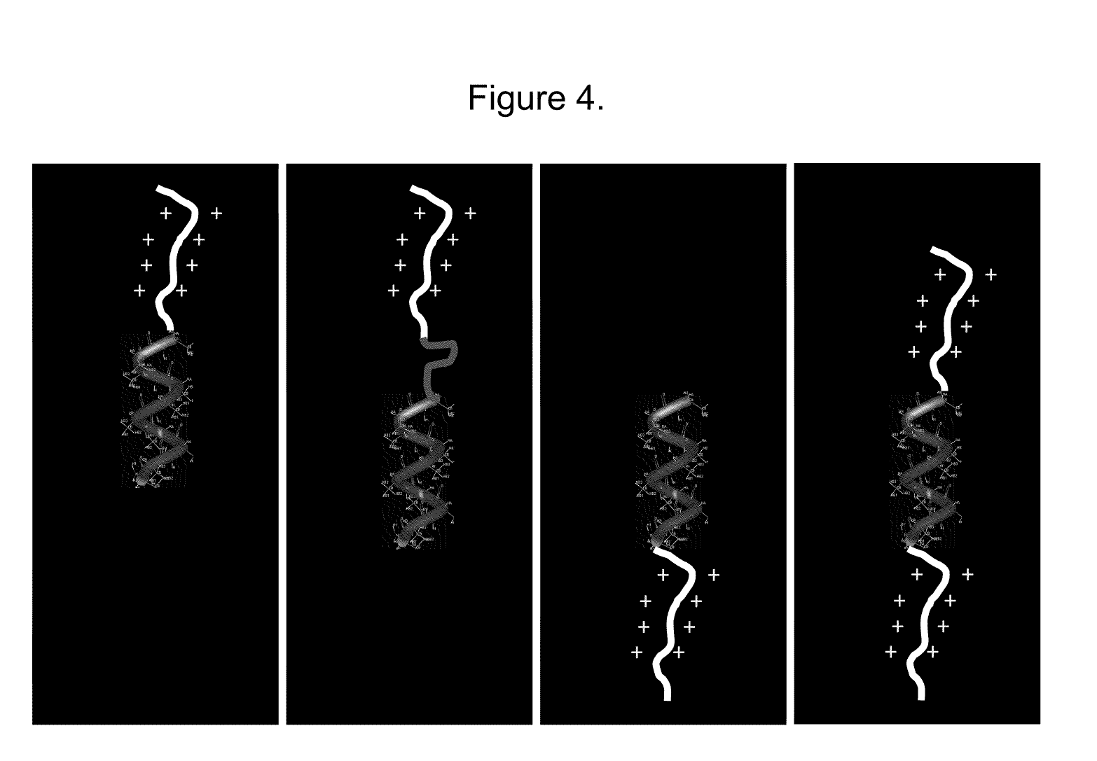

To evaluate the mechanism of action of the invention, a high dose of BoNT/A (50,000 LD50) was administered to two groups of mice using BoNT/A coated on microneedles as described above. Hyaluronidase was added to the microneedles used for one of the groups, also as indicated previously. The mice were monitored and the lethality of BoNT/A was determined at 24 and 48 hours. The results are shown in FIG. 2, indicating that the microneedles coated with hyaluronidase allowed faster onset of the full activity of botulinum neurotoxin relative to microneedles not coated with hyaluronidase.

### Example 3

### Application of the Invention to Treatment of Glabella, Forehead or Nasolabial Fold Wrinkles in a Patient

Microneedle arrays are coated with BoNT/B and hyaluronidase and are applied to a patient's forehead, glabella and/or nasolabial fold for 60 seconds. The hyaluronidase deposited in the tissue begins to digest extracellular hyaluronic acid in the skin and subcutaneous tissue within 3 minutes. Due to the presence of hyaluronidase, the dose of BoNT/B delivered is lower to achieve the same muscle paralysis, as compared with BoNT/B delivered on microneedles without hyaluronidase. Relief from wrinkles is observed in the patient.

### Example 4

### Application of the Invention to Treatment of Palmar Hyperhidrosis

Uncoated solid microneedle arrays (i.e., without any agent) are repeatedly applied against a patient's palms to perforate and create microchannels through the stratum corneum into the dermal layer of skin. This is followed by topical application of a formulation comprising hyaluronidase and BoNT/B on the surface of the palms. The patients exhibits decreased sweating after treatment, comparable to treatment with intradermal multiple injections with hypodermic needles on each palm, and requires a lower BoNT/B dose than topical application without microneedle pre-poration.

### Example 5

### Application of the Invention to Treatment of Axillar Hyperhidrosis

Microneedle arrays coated with hyaluronidase are applied to a patient's axilla to perforate and create microchannels through the stratum corneum into the dermal layer of skin. This delivers hyaluronidase into the skin and resulting in increased breakdown of extracellular matrix for enhanced BTX transport. This will be followed by topical application of BTX on the surface of the palms. The patients will exhibit decreased sweating after treatment, comparable to treatment with intradermal multiple injections with hypodermic needles in each axilla, and will require lower BTX dose than topical application without microneedle pre-poration and hyaluronidase pre-treatment.

### Example 6

### Use of a Targeting Moiety for Enhancing Delivery of Botulinum Neurotoxin to Tissue

A targeting moiety in addition to botulinum neurotoxin are coated on the same microneedle array.

The microneedle array is applied to the glabella, forehead and nasolabial folds, as required for each site. The presence of the targetin moiety result in improved efficacy using lower doses of botulinum neurotoxin, as compared with botulinum neurotoxin delivered on microneedles in the absence of targeting moiety.

### Example 7

### Use of a Gel Formulation for Delivering Botulinum Neurotoxin and a Targeting Moiety

A targeting moiety in combination with botulinum neurotoxin is formulated as a gel and is applied topically on the palms of a patient with hyperhidrosis. The agents is left on the patient's palms for 5 to 15 minutes, before being wiped off. The topical botulinum neurotoxin dose needed for obtaining efficacy is be reduced relative to topical botulinum neurotoxin application without targeting moieties.

### Example 8

### Synthesis of Targeting Moieties for Delivery of Botulinum Neurotoxins

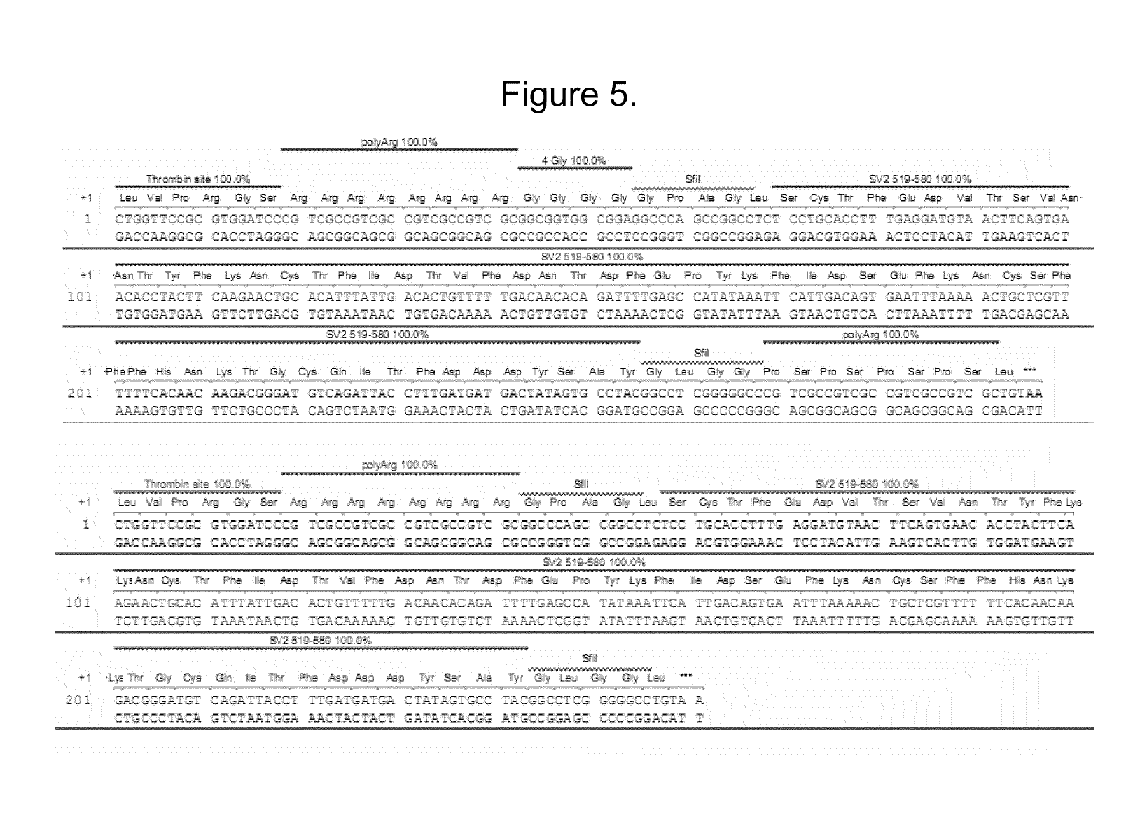

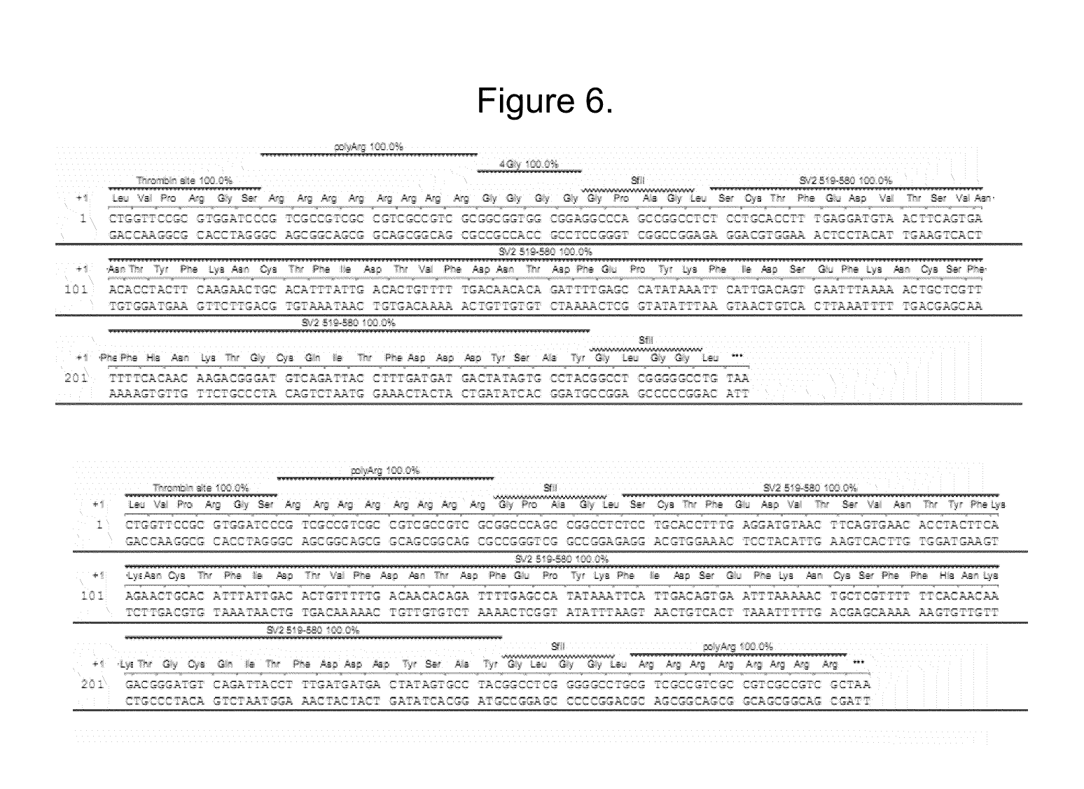

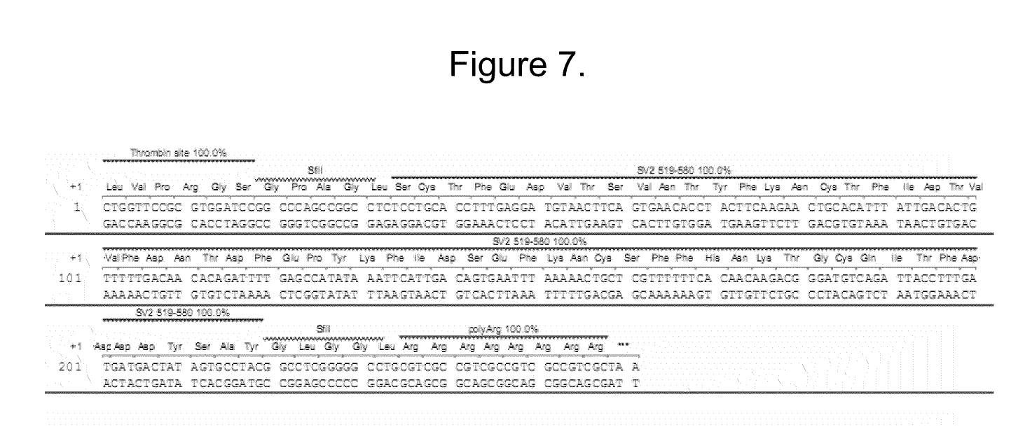

Several targeting moieties were synthesized using general molecular biology techniques and the resulting nucleotides and peptide sequences are described in FIGS. 5-7. Generally, the targeting moieties prepared included a botulinum neurotoxin binding site comprising amino acids 519-580 of the SV2 protein, a transport moiety which is a polyArg sequence, and/or a flexible Gly₄ linker. “Sfil” indicates an Sfil restriction endonuclease recognition sequence, while “thrombin site” represents a thrombin protease cleavage site.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A microneedle device comprising one or more microneedles that:
(a) is coated with up to 1,000 units of a botulinum neurotoxin;
(b) contains up to 1,000 units of a botulinum neurotoxin; or
(c) is effective to deliver up to 1,000 units of a botulinum neurotoxin per injection,
wherein the microneedle device:
(a) is coated with a formulation comprising an enzyme system which digests an extracellular matrix;
(b) contains within the needle a formulation comprising an enzyme system that digests an extracellular matrix and a botulinum neurotoxin; or
(c) is effective to deliver an enzyme that digests an extracellular matrix.

2. The microneedle device of claim 1, wherein said botulinum neurotoxin is associated with a targeting moiety.

3. The microneedle device of claim 3, wherein the targeting moiety comprises a transport moiety.

4. The microneedle device of claim 1, wherein the botulinum neurotoxin resides essentially only on the tip of the one or more microneedles.

5. A method for treating a skin region having wrinkles comprising:
(a) applying a microneedle device comprising one or more microneedles to the skin region to perforate the skin region;
(b) applying to the skin region a penetration enhancer; and
(c) applying to the skin region a botulinum neurotoxin.

6. The method of claim 5, wherein steps (b) and (c) occur simultaneously.

7. The method of claim 5, wherein steps (a), (b) and (c) occur simultaneously.

8. The method of claim 5, wherein step (b) or (c) occurs using a topical patch.

9. The method of claim 5, wherein step (b) or (c) occurs using a subcutaneous injection.

10. The method of claim 5, wherein step (a) occurs prior to step (b) and step (c).

11. The method of claim 5, wherein step (b) and step (c) are performed using a microneedle device.

12. The method of claim 5, wherein step (a), (b), or (c) is applied for at least 20 seconds, 25 seconds, 30 seconds or 60 seconds.

13. The method of claim 12, wherein the microneedle device is dissolvable.

14. The method of claim 5, wherein step (b) and step (c) are performed using topical administration of a foam or a gel.

15. The method of claim 5, wherein the penetration enhancer comprises an enzyme that degrades extracellular matrix.

16. The method of claim 15, wherein the enzyme is selected from the group consisting of papain, hyaluronidase, streptokinase, streptodornase, trypsin, chymotrypsin, alpha-chymotrypsin, alpha-amylase, deoxyribonuclease, collagenase, sutilain, and any mixture thereof.

17. The method of claim 5, wherein the step of applying a penetration enhancer comprises applying papain, Azone™, and propylene glycol.

18. The method of claim 5, wherein step (a), (b) or (c) are repeated at least 2, 3, 4, 5, 6, 7, or 8 times.

19. The method of claim 5, wherein steps (a), (b) and (c) are repeated at least 2, 3, 4, 5, 6, 7, or 8 times.

20. The method of claim 18 or 19, wherein the repetition is per month or per year.

21. A method for treating a skin region having wrinkles comprising administering to the skin region less than 100, 500, 1000, 2000, 4000 or 10000 units of a botulinum neurotoxin, wherein the administering is performed by applying a microneedle device to the skin region.

22. The method of claim 21, further comprising administering to the skin region an enzyme that digests an extracellular matrix.

23. The method of claim 21, further comprising perforating the skin region by applying to it said microneedle device.

24. The method of claim 6 or 21, wherein the skin region is a forehead, a palm, glabella, or nasolabial folds.

25. The method of claim 21, wherein the administering step occurs for at least at least 20 seconds, 25 seconds, 30 seconds or 60 seconds.

26. An injectable formulation consisting essentially of a botulinum neurotoxin and a penetration enhancer.

27. The formulation of claim 26, wherein the penetration enhancer comprises papain and optionally Azone™ and propylene glycol.

28. The formulation of claim 26, wherein the penetration enhancer is an enzyme that degrades extracellular matrix.

29. The formulation of claim 28, wherein the enzyme is selected from the group consisting of: papain, hyaluronidase, streptokinase, streptodornase, trypsin, chymotrypsin, alpha-chymotrypsin, alpha-amylase, deoxyribonuclease, collagenase, sutilain and any mixture thereof.

30. The formulation of claim 26, wherein said botulinum neurotoxin is serotype A, B, C1, D, E, F, or G.

31. The formulation of claim 26, wherein said botulinum neurotoxin is associated with a targeting moiety.

32. The formulation of claim 31, wherein said targeting moiety comprises a polyarginine.

33. A targeting moiety construct comprising:
(a) a transport moiety for delivering a load across the skin barrier; and
(b) a botulinum neurotoxin binding moiety linked to said transport moiety.

34. The construct of claim 33, further comprising a second transport moiety linked to said botulinum neurotoxin binding moiety.

35. The construct of claim 33, wherein the transport moiety is a dendrimer or a polyarginine.

36. The construct of claim 33, wherein the botulinum neurotoxin binding moiety is SV2 (518-580) or Syt II (40-60).

37. The construct of claim 33, further comprising a flexible linker.

38. The construct of claim 37, wherein the linker is Gly-Ser.

39. The construct of claim 33, wherein the botulinum neurotoxin binding moiety is an antibody.

40. The construct of claim 33, wherein the botulinum neurotoxin binding moiety is a botulinum neurotoxin binding peptide or a botulinum neurotoxin binding antibody.

41. An injectable formulation comprising the construct of claim 33.

42. A method for making a microneedle device comprising:
(a) providing a plurality of integrated microneedles;
(b) coating at least one or more of the microneedles with a botulinum neurotoxin or an extracellular matrix degrading enzyme; and
(c) optionally further contacting at least one or more of the microneedles with a penetration enhancer.

43. The method of claim 42, wherein the microneedles are degradable.

44. The method of claim 42, wherein the penetration enhancer is selected from the group consisting of: papain, hyaluronidase, streptokinase, streptodornase, trypsin, chymotrypsin, alpha-chymotrypsin, alpha-amylase, deoxyribonuclease, collagenase, sutilain and any mixture thereof.

45. A kit consisting essentially of a microneedle device, a botulinum neurotoxin system and a penetration enhancer.

46. The kit of claim 45, wherein the microneedle device comprises one or more porous microneedles.

47. The kit of claim 45, wherein the microneedle device comprises one or more non-porous microneedles.

48. The kit of claim 45, wherein the microneedle device comprises a reservoir attached to one or more microneedles.

49. The kit of claim 45, wherein the penetration enhancer is an enzyme system that digests extracellular matrix.

50. The kit of claim 49, wherein the enzyme system comprises a protease or a hyaluronidase.

51. The kit of claim 49, wherein the protease is a matrix metalloprotease, a thiol protease or a serine protease.

52. The kit of claim 45, wherein the botulinum neurotoxin system comprises botulinum neurotoxin A.

53. The kit of claim 45, wherein the botulinum neurotoxin system comprises botulinum neurotoxin B.

54. The kit of claim 45, wherein either the enzyme system or the botulinum neurotoxin system is coated on a subset of the microneedles.

55. The kit of claim 45, wherein either the enzyme system or the botulinum neurotoxin system is within a subset of the microneedles.

56. The kit of claim 45, wherein the botulinum neurotoxin system comprises a botulinum neurotoxin associated with a targeting moiety.

57. The kit of claim 56, wherein the targeting moiety comprises a transport moiety which is a poly-arginine.

58. The kit of claim 49, wherein either the enzyme system or the botulinum neurotoxin system is formulated in a patch.

59. The kit of claim 49, wherein either the enzyme system or the botulinum neurotoxin system is formulated for subcutaneous or subdermal delivery via injection.

60. The kit of claim 45, wherein the microneedle device comprises dissolvable microneedles.

61. The kit of claim 45, wherein the botulinum neurotoxin is BOTOX™.

62. The kit of claim 61, wherein the microneedle device is configured for delivering at least 4, 50, 100, 400, 1000, or 2000 units of BOTOX™.

63. The kit of claim 45, wherein the botulinum neurotoxin resides essentially only on the tip of microneedle(s) in the microneedle device.


SEQUENCE LISTING (33 sequences)

SEQ ID NO: 1 - protein, 11 aa, Human immunodeficiency virus
YGRKKRRQRRR

SEQ ID NO: 2 - protein, 7 aa, Human immunodeficiency virus
KRRQRRR

SEQ ID NO: 3 - protein, 7 aa, Human immunodeficiency virus
RKKRRQR

SEQ ID NO: 4 - protein, 8 aa, Human immunodeficiency virus
RKKRRQRR

SEQ ID NO: 5 - protein, 4 aa, Artificial Sequence
  Description of Artificial Sequence Synthetic peptide
RRRR

SEQ ID NO: 6 - protein, 5 aa, Artificial Sequence
  Description of Artificial Sequence Synthetic peptide
RRRRR

SEQ ID NO: 7 - protein, 16 aa, Artificial Sequence
  Description of Artificial Sequence Synthetic peptide
RRRRRRRRRRRRRRRR

SEQ ID NO: 8 - protein, 16 aa, Unknown
  Description of Unknown Yeast PRP peptide
TRRNKRNRIQEQLNRK

SEQ ID NO: 9 - protein, 18 aa, Unknown
  Description of Unknown Phi21 peptide
TAKTRYKAEEAELIAERR

SEQ ID NO: 10 - protein, 22 aa, Unknown
  Description of Unknown LambdaN peptide
MDAQTRRRERRAEKQAQWKAAN

SEQ ID NO: 11 - protein, 15 aa, Feline herpes virus
RRRRNRTRRNRRRVR

SEQ ID NO: 12 - protein, 6 aa, Artificial Sequence
  Description of Artificial Sequence Synthetic peptide
RRRRRR

SEQ ID NO: 13 - protein, 19 aa, Brome mosaic virus
KMTRAQRRAAARRNRWTAR

SEQ ID NO: 14 - protein, 13 aa, Human T-lymphotropic virus
TRRQRTRRARRNR

SEQ ID NO: 15 - protein, 17 aa, Human immunodeficiency virus
TRQARRNRRRRWRERQR

SEQ ID NO: 16 - protein, 13 aa, Human immunodeficiency virus
GRKKRRQRRRPPQ

SEQ ID NO: 17 - protein, 9 aa, Human immunodeficiency virus
RRRQRRKKR

SEQ ID NO: 18 - protein, 9 aa, d-Human immunodeficiency virus
RKKRRQRRR

SEQ ID NO: 19 - protein, 7 aa, Artificial Sequence
  Description of Artificial Sequence Synthetic peptide
RRRRRRR

SEQ ID NO: 20 - protein, 7 aa, Artificial Sequence
  Description of Artificial Sequence Synthetic peptide
RRRRRRR

SEQ ID NO: 21 - protein, 8 aa, Artificial Sequence
  Description of Artificial Sequence Synthetic peptide
RRRRRRRR

SEQ ID NO: 22 - protein, 9 aa, Artificial Sequence
  Description of Artificial Sequence Synthetic peptide
RRRRRRRRR

SEQ ID NO: 23 - protein, 4 aa, Artificial Sequence
  Description of Artificial Sequence Synthetic peptide
GGGG

SEQ ID NO: 24 - DNA, 297 bp, Artificial Sequence
  Description of Artificial Sequence Synthetic polynucleotide
ctggttccgcgtggatcccgtcgccgtcgccgtcgccgtcgcggcggtggcggaggcccagccggcctctcctgcacctttgaggatgtaacttcagtgaacacctacttcaagaactgcacatttattgacactgtttttgacaacacagattttgagccatataaattcattgacagtgaatttaaaaactgctcgttttttcacaacaagacgggatgtcagattacctttgatgatgactatagtgcctacggcctcgggggcccgtcgccgtcgccgtcgccgtcgctgtaa

SEQ ID NO: 25 - protein, 98 aa, Artificial Sequence
  Description of Artificial Sequence Synthetic polypeptide
LVPRGSRRRRRRRRGGGGGPAGLSCTFEDVTSVNTYFKNCTFIDTVFDNTDFEPYKFIDSEFKNCSFFHNKTGCQITFDDDYSAYGLGGPSPSPSPSL

SEQ ID NO: 26 - DNA, 261 bp, Artificial Sequence
  Description of Artificial Sequence Synthetic polynucleotide
ctggttccgcgtggatcccgtcgccgtcgccgtcgccgtcgcggcccagccggcctctcctgcacctttgaggatgtaacttcagtgaacacctacttcaagaactgcacatttattgacactgtttttgacaacacagattttgagccatataaattcattgacagtgaatttaaaaactgctcgttttttcacaacaagacgggatgtcagattacctttgatgatgactatagtgcctacggcctcgggggcctgtaa

SEQ ID NO: 27 - protein, 86 aa, Artificial Sequence
  Description of Artificial Sequence Synthetic polypeptide
LVPRGSRRRRRRRRGPAGLSCTFEDVTSVNTYFKNCTFIDTVFDNTDFEPYKFIDSEFKNCSFFHNKTGCQITFDDDYSAYGLGGL

SEQ ID NO: 28 - DNA, 273 bp, Artificial Sequence
  Description of Artificial Sequence Synthetic polynucleotide
ctggttccgcgtggatcccgtcgccgtcgccgtcgccgtcgcggcggtggcggaggcccagccggcctctcctgcacctttgaggatgtaacttcagtgaacacctacttcaagaactgcacatttattgacactgtttttgacaacacagattttgagccatataaattcattgacagtgaatttaaaaactgctcgttttttcacaacaagacgggatgtcagattacctttgatgatgactatagtgcctacggcctcgggggcctgtaa

SEQ ID NO: 29 - protein, 90 aa, Artificial Sequence
  Description of Artificial Sequence Synthetic polypeptide
LVPRGSRRRRRRRRGGGGGPAGLSCTFEDVTSVNTYFKNCTFIDTVFDNTDFEPYKFIDSEFKNCSFFHNKTGCQITFDDDYSAYGLGGL

SEQ ID NO: 30 - DNA, 285 bp, Artificial Sequence
  Description of Artificial Sequence Synthetic polynucleotide
ctggttccgcgtggatcccgtcgccgtcgccgtcgccgtcgcggcccagccggcctctcctgcacctttgaggatgtaacttcagtgaacacctacttcaagaactgcacatttattgacactgtttttgacaacacagattttgagccatataaattcattgacagtgaatttaaaaactgctcgttttttcacaacaagacgggatgtcagattacctttgatgatgactatagtgcctacggcctcgggggcctgcgtcgccgtcgccgtcgccgtcgctaa

SEQ ID NO: 31 - protein, 94 aa, Artificial Sequence
  Description of Artificial Sequence Synthetic polypeptide
LVPRGSRRRRRRRRGPAGLSCTFEDVTSVNTYFKNCTFIDTVFDNTDFEPYKFIDSEFKNCSFFHNKTGCQITFDDDYSAYGLGGLRRRRRRRR

SEQ ID NO: 32 - DNA, 261 bp, Artificial Sequence
  Description of Artificial Sequence Synthetic polynucleotide
ctggttccgcgtggatccggcccagccggcctctcctgcacctttgaggatgtaacttcagtgaacacctacttcaagaactgcacatttattgacactgtttttgacaacacagattttgagccatataaattcattgacagtgaatttaaaaactgctcgttttttcacaacaagacgggatgtcagattacctttgatgatgactatagtgcctacggcctcgggggcctgcgtcgccgtcgccgtcgccgtcgctaa

SEQ ID NO: 33 - protein, 86 aa, Artificial Sequence
  Description of Artificial Sequence Synthetic polypeptide
LVPRGSGPAGLSCTFEDVTSVNTYFKNCTFIDTVFDNTDFEPYKFIDSEFKNCSFFHNKTGCQITFDDDYSAYGLGGLRRRRRRRR